(19) United States
(12) Patent Application Publication
(10) Pub. No.: US 2006/0150734 A1
(43) Pub. Date: Jul. 13, 2006

(54) Activity monitoring

(75) Inventors: Gillian Antoinette Mimnagh-Kelleher, Eindhoven (NL); Paraskevas Dunias, Eindhoven (NL); Joannes Gregorius Bremer, Eindhoven (NL); Adrianus Petrus Johanna Maria Rommers, Eindhoven (NL); Wilhelmus Lambertus Marinus Cornelius Verhoeven, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., Eindhoven (NL)

(21) Appl. No.: 10/537,877

(22) PCT Filed: Nov. 21, 2003
(86) PCT No.: PCT/IB03/05333

§ 371 Date: Jun. 7, 2005

(30) Foreign Application Priority Data
Dec. 10, 2002 (EP) 02080214.6

(51) Int. Cl. A61B 5/11

(52) U.S. Cl. 73/510; 702/189; 73/509; 600/595

(57) ABSTRACT

An activity monitor is provided that operates to reduce errors associated with current activity monitors caused by simply adding activity values from different motion sensors.

The present invention relates to activity monitoring, and in particular, but not exclusively to, activity monitoring of a human being.

The physical activity of a human being is an important determinant of its health. The amount of daily physical activity is considered to be a central factor in the etiology, prevention and treatment of various diseases. Information about personal physical activity can assist the individual in maintaining or improving his or her functional health status and quality of life.

A known system for monitoring human activity is described in the article “A Triaxial Accelerometer and Portable Data Processing Unit for the Assessment of Daily Physical Activity”, by Bouten et al., IEEE Transactions on Biomedical Engineering, Vol. 44, NO.3, March 1997.

According to the known system a triaxial accelerometer composed of three orthogonally mounted uniaxial piezoresistive accelerometers is used to measure accelerations covering the amplitude and frequency ranges of human body acceleration. An individual wears the triaxial accelerometer over a certain period of time. A data processing unit is attached to the triaxial accelerometer and programmed to determine the time integrals of the moduli of accelerometer output from the three orthogonal measurement directions. These time integrals are summed up and the output is stored in a memory that can be read out by a computer. The output of the triaxial accelerometer bears some relation to energy expenditure due to physical activity and provides as such a measure for the latter.

The known system allows for measurement of human body acceleration in three directions. Using state of the art techniques in the field of integrated circuit technology the accelerometer can be built small and lightweight allowing it to be worn for several days or even longer without imposing a burden to the individual wearing it.

However, the known system has the considerable drawback that simply adding the outputs of the respective accelerometers means that errors are introduced for movements that are not paraxial. For example, for movements which lie in the z plane between the x and y axes, the maximum error is √2 (approximately 41%). For the three axis, the error can be as high as √3 (approximately 73%).

It is therefore desirable to provide an activity monitor that can overcome these disadvantages.

According to one aspect of the present invention, there is provided an activity monitor comprising a measurement unit including a plurality of motion sensors for producing respective sensor signals indicative of motion experienced thereby; a processor operable to receive the sensor signals from the measurement unit, and to process the sensor signals in accordance with a predetermined method, characterized in that the processor is operable to process the sensor signals as respective vector components.

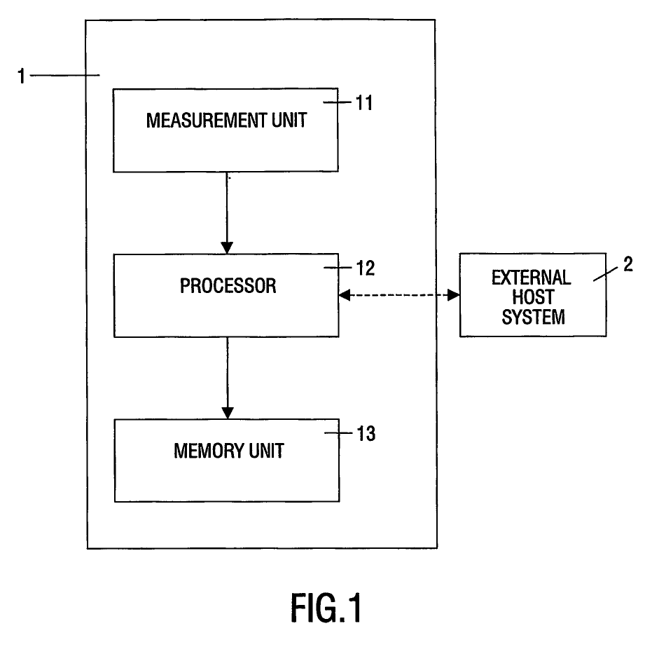

FIG. 1 shows a block diagram schematically showing the components of a system embodying one aspect of the present invention;

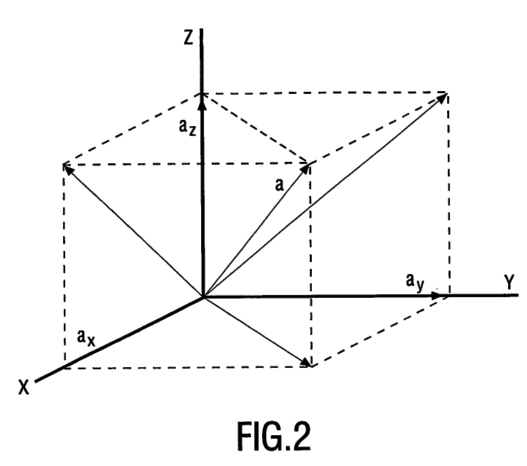

FIG. 2 schematically shows the orthogonal position of three accelerometers; and

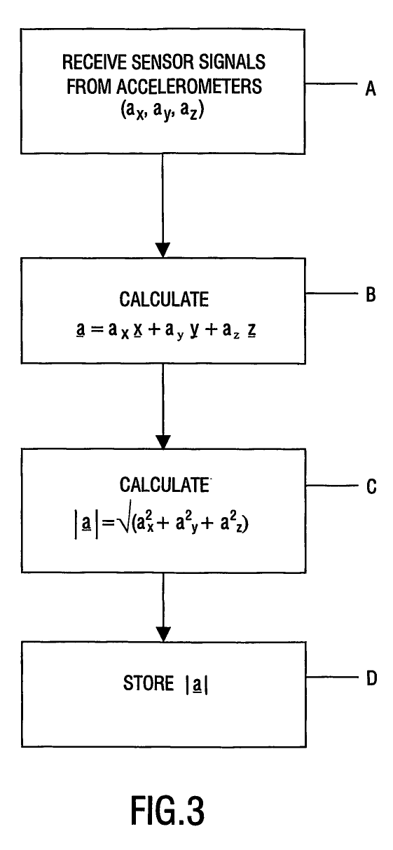

FIG. 3 shows a flow diagram of the steps of a method embodying another aspect of the present invention.

FIG. 1 illustrates an activity monitor 1 embodying one aspect of the present invention. The activity monitor 1 comprises a measurement unit 11, a processor 12, and a memory unit 13. The measurement unit 11 is operable to produce data signals indicative of the motion of the activity monitor 1, and to supply those data signals to the processor 12. The processor 12 is operable to process the data signals output from the measurement unit, and is able to store the data signals, or the results of the processing in the memory unit 13. Data can be transferred between the processor and the memory unit 13. The processor 12 is also able to be connected to an external hose system 2, which can be a personal computer (PC) or other appropriate systems. The external hose system 2 can be used to perform additional processing of the data held in the activity monitor 1.

In use, the activity monitor 1 is attached to the object to be monitored. For purposes of illustration in the following it is assumed that the object is a human individual, although it is clearly possible to apply such an activity monitor for any object. The activity monitor is attached to the individual or object for a certain time period.

The measurement unit comprises three accelerometers which are arranged in mutually orthogonal directions. The accelerometers output data signals which are indicative of the respective accelerations experienced by the accelerometers. The three accelerometers are arranged orthogonal to one another in a conventional manner.

On an individual, these directions are formed “antero-posterior”, “medio-lateral” and “vertical”, that are denoted as x, y and z, respectively. The accelerometers comprise strips of piezo-electric material that is uni-axial and serial bimorph. The strips are fixed at one end thereof.

The piezo-electric accelerometers act as damped mass-spring systems, wherein the piezo-electric strips act as spring and damper. Movements of the strips due to movement of the individual generate an electric charge leading to a measurement of a data signal. In case of human movements the frequency of the data signals lies in the range of 0.1-20 Hz. The amplitude of the data signals lies between −12 g and +12 g. These numbers are discussed in more detail in the article mentioned earlier. Suitable piezo-electric materials to measure such data signals are known to a person skilled in the art.

FIG. 2 illustrates the-orthogonal output of the three accelerometers of the measurement unit 11. The outputs are termed aₓ, a_{y} and a_{z} respectively. In accordance with the present invention, these output data signals from the accelerometers are treated as orthogonal components of an acceleration vector a. Accordingly, the magnitude of the vector a is known to be a=√(aₓ²+a_{y}²+a_{z}²). Treating the outputs of the accelerometers in this way, and thereby calculating the magnitude of the vector a, enables the previously generated errors to be corrected. Thus, the magnitude of vector a gives an accurate reflection of the summed accelerations experienced by the activity monitor 1. In addition, the acceleration vector a automatically then includes some direction information regarding the net acceleration measured by the accelerometers.

Although calculating the magnitude of vector a could be a processor intensive activity, in a preferred embodiment of the present invention, a lookup table is provided giving the magnitude of a for various different values of aₓ, a_{y} and a_{z}. Thus calculation of the magnitude of a can be achieved simply by a table lookup. The use of a lookup table can enable lower power consumption, since the lookup operation is more efficient than using an algorithm to calculate the required result. Typically, the accuracy of the result needs to be of the order of +/−1%, and so the data to be stored is fairly limited. This has the advantage that only limited memory resources are required to supply the required results.

For the sake of clarity, FIG. 3 illustrates a method embodying another aspect of the present invention. At step A, the processor receives data signals from the three accelerometers. The vector a is calculated at step B using the data signals, and the magnitude of vector a is calculated at step C. As discussed above, the calculation can be made by the processor directly, or can be made by a table lookup process. At step D, the resulting magnitude of a is stored in the memory unit 13. In addition, information relating to the direction of a can be stored in memory.

It will be readily appreciated that the accelerometers are merely preferred motion sensors, and that any appropriate motion sensor could be used in an embodiment of the present invention and achieve the advantages of the present invention.

It will therefore be appreciated that an activity monitor and method embodying the present invention are able to correct for errors created in the previously considered activity monitors. It is emphasised that the term “comprises” or “comprising” is used in this specification to specify the presence of stated features, integers, steps or components, but does not preclude the addition of one or more further features, integers, steps or components, or groups thereof.

1. An activity monitor comprising:
a measurement unit including a plurality of motion sensors for producing respective sensor signals indicative of motion experienced thereby; and
a processor operable to receive the sensor signals from the measurement unit, and to process the sensor signals in accordance with a predetermined method,
characterized in that the processor is operable to process the sensor signals as respective vector components to produce a resultant vector.

2. An activity monitor as claimed in claim 1, wherein the motion sensors are accelerometers.

3. An activity monitor as claimed in claim 1, wherein the motion sensors are arranged to be mutually orthogonal.

4. An activity monitor as claimed in claim 3, wherein the processor is operable to calculate the magnitude of the resultant vector according to the following expression:
a=√(aₓ²+a_{y}²+a_{z}²),
where a is the magnitude of the resultant vector, aₓ, a_{y} and a_{z} are respective sensor signals.

5. An activity monitor as claimed in claim 4, wherein values of a are stored in a lookup table.

6. An activity monitor as claimed in claim 4, wherein the processor is operable to calculate the direction of the resultant vector.

7. A method of monitoring activity using a plurality of motion sensors which are operable to produce respective sensor signals indicative of motion experienced thereby, the method comprising receiving sensor signals and processing the signals in accordance with a predetermined method, characterized in that the sensor signals are processed as respective vector components to produce a resultant vector.

8. A method as claimed in claim 7, wherein the magnitude of the resultant vector according to the following expression:
a=√(aₓ²+a_{y}²+a_{z}²),
where a is the magnitude of the resultant vector, aₓ, a_{y} and a_{z} are respective sensor signal.

9. A method as claimed in claim 7, comprising calculating and storing the direction of the resultant vector.
